(19) United States
(12) Patent Application Publication
(10) Pub. No.: US 2026/0354860 A1
(43) Pub. Date: Nov. 12, 2026

(54) GASTRIC CALIBRATION TUBE

(71) Applicant: Q MEDICAL INTERNATIONAL AG, Stein am Rhein (CH)

(72) Inventor: Arnoldinus Carolus Martijn VAN DER VEEK, Amsterdam (NL)

(73) Assignee: Q MEDICAL INTERNATIONAL AG, Stein am Rhein (CH)

(21) Appl. No.: 19/475,212

(22) PCT Filed: Apr. 25, 2023
(86) PCT No.: PCT/EP2023/060843

§ 371 Date: Oct. 14, 2025

(51) Int. Cl. A61B 17/34; A61B 17/00

(52) U.S. Cl. CPC A61B 17/3415; A61B 2017/00818

(57) ABSTRACT

A gastric calibration tube having an elongate flexible hollow body which has an outer wall and a first and a second tube end. The elongate flexible hollow body has at least one opening in the region of the second tube end, the ratio of the wall thickness of the elongate flexible hollow body to the outer diameter of the elongate flexible hollow body is between 0.1 and 0.25.

### FIELD

The present invention relates to a gastric calibration tube.

### BACKGROUND

Gastric calibration tubes are known from prior art, which can be used in surgical procedures, in particular in gastric and bariatric procedures such as gastric resections, laparoscopic banded sleeve gastrectomy (LBSG), conventional sleeve gastrectomy (LCSG), fundoplication, gastric band insertion and, for example, gastric bypasses. The gastric calibration tube is used to visibly and palpably delineate parts of the stomach, to introduce air or liquid (methylene blue) and to decompress, empty and remove liquids.

The gastric calibration tube has an elongate flexible hollow body with an outer wall and a first and a second tube end. The second end of the tube can be inserted into the stomach via the mouth and esophagus. The first end of the tube remains outside the body. Medical aids, e.g. devices such as syringes, cannulas or devices that introduce or discharge air or liquids into the calibration tube, can be connected to the first end of the tube. Discharge can take place, for example, by creating a negative pressure in the calibration tube.

### SUMMARY

The object of the present invention is to provide a gastric calibration tube which is further improved.

The invention advantageously provides that the ratio between the wall thickness of the elongate flexible hollow body in relation to the outer diameter of the elongate flexible hollow body is between 0.1 and 0.25.

The present invention has the advantage that the gastric calibration tube does not kink if the wall thickness is appropriate in relation to the outer diameter of the hollow body.

If a tube kinks, this means that the flow of liquids or gases in the tube is disrupted or completely blocked. Kinking can also damage the calibration tube. Further, a result of kinking can be that the second end of the tube cannot be guided to the anatomical target, e.g. the transition from the stomach to the duodenum.

With the geometric proportions of the present gastric calibration tube according to the invention, kinking no longer or hardly ever occurs.

The elongate flexible hollow body can be made of a material containing silicone, preferably silicone rubber. The material, in particular the silicone rubber, can preferably have a Shore hardness A of between 60 and 100.

Due to the features according to the invention, the gastric calibrating tube is very difficult to cut by a surgical stapler and/or is very difficult to staple over.

Shore hardness testing is a simple and effective method of material testing and a simple procedure for measuring the hardness of elastomers and deformable base materials. Shore hardness is the resistance of a sample to the penetration of a conical body of certain dimensions under a defined compressive force. Depending on the design of the measuring body, a distinction is made between Shore A and Shore D. In this case, the specifications refer to the Shore A hardness.

A detailed description of how the hardness test is to be carried out is given in DIN ISO 7619/1.

In particular, the silicone rubber has a Shore hardness A of between 70 and 90.

The flexible hollow body is elongate and has a longitudinal axis. The hollow body is therefore longer in a direction parallel to the longitudinal axis than it is wide in a direction transverse to the longitudinal axis.

The term “in the area of the second tube end” is used in the present invention, whereby the term is to be understood as meaning that this area comprises at least the half of the calibration tube that also comprises the second tube end. Preferably, “in the range of” is to be understood as meaning that only ⅓, in particular ¼, especially preferably ⅕ of the calibration tube comprises the respective tube end.

The same applies to the use of the term “in the area of the first tube end”.

The elongate flexible hollow body can have at least such a resistance to kinking that with a tube section of maximum 20 cm in length, the two tube section ends can be brought together and the tube section does not kink.

The elongate flexible hollow body can further have at least such a resistance to kinking that with a tube section of maximum 15 cm in length, the two tube section ends can be brought together and the tube section does not kink.

Kinking resistance means the resistance or resistance of the hollow body to kinking.

The ratio between the wall thickness of the elongate flexible hollow body in relation to the outer diameter of the elongate flexible hollow body is between 0.16 and 0.2.

Outlet openings can be provided in the region of the second tube end.

A terminating element can be provided at the second end of the tube to close off the hollow body.

The terminating element can be blunt. In this way, the gastric calibration tube can be easily inserted into the stomach via the esophagus without damaging tissue.

The terminating element can include a material that comprises radiopaque material, preferably barium sulphate.

In this way, the terminating element can be localized using X-ray equipment, for example. This can ensure that the calibration tubes are not lost in the body during surgery.

The terminating element can have at least one outlet opening.

The term flexible hollow body is used in the present invention. In this context, flexible means that the hollow body can be bent. It should preferably be able to be bent by at least 90°.

The material from which the terminating element is made can be softer than the material from which the elongate flexible hollow body is made.

A material is softer if it deforms more quickly. For example, it deforms more quickly under pressure or other loads.

The material from which the cover element is made can comprise silicone, preferably silicone rubber.

In the longitudinal direction of the elongate flexible hollow body, at least one color strip can be arranged along the elongate flexible hollow body.

The color strip may differ in material and/or color from the elongate flexible hollow body.

The color strip can be recessed in the elongate flexible hollow body so that the surface of the color strip is flush with the outer surface of the elongate flexible hollow body.

The color strip can have a thickness that does not exceed the wall thickness of the elongate flexible hollow body. Thus, the color strip cannot penetrate the wall thickness of the hollow body.

The elongate flexible hollow body can have first outlet openings on a first side of the hollow body in the region of the second tube end and second outlet openings on a second side of the hollow body opposite the first side.

In the following, an embodiment of the present invention will be described in more detail with reference to the Figures.

### BRIEF DESCRIPTION OF THE FIGURES

The Figures show schematically:

FIG. 1a a gastric calibration tube according to the present invention,

FIG. 1b a locking slider for closing the calibration tube,

FIG. 2 a section of the gastric calibration tube with outlet opening,

FIG. 3 the section from FIG. 2 in a different side view and with color strip,

FIG. 4 a cross-section of the gastric calibration tube,

FIG. 5 a longitudinal cross-section of a section of the gastric calibration tube,

FIG. 6 another view of a section of the gastric calibration tube.

### DETAILED DESCRIPTION

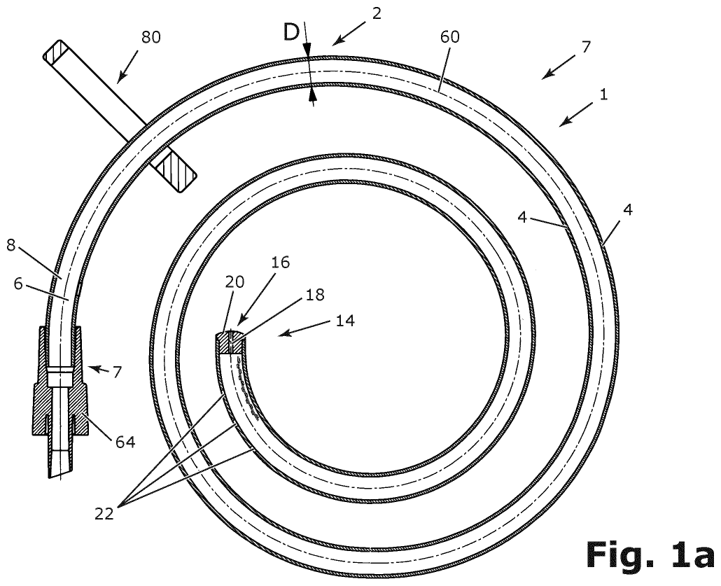

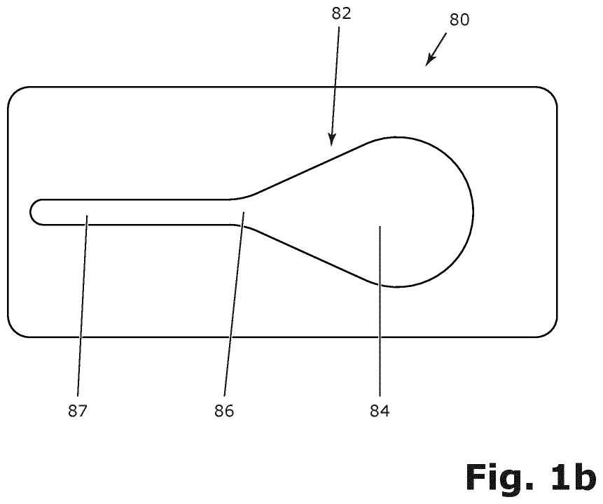

FIG. 1a shows a gastric calibration tube 1. The gastric calibration tube 1 has an elongate flexible hollow body 2. The elongate flexible hollow body 2 has a longitudinal axis 60. Furthermore, the hollow body 2 has an outer wall 4. The hollow body 2 has a first tube end 7 and a second tube end 14. The second end 14 of the tube can be inserted into the stomach via the mouth and esophagus. The first end 7 of the tube remains outside the body.

The gastric calibration tube 1 can be used in surgical procedures, in particular in gastric and bariatric procedures such as gastric resections, laparoscopic banded sleeve gastrectomy (LBSG), conventional sleeve gastrectomy (LCSG), fundoplication, gastric band insertion and, for example, gastric bypasses. The gastric calibration tube 1 is used to visibly and palpably delineate parts of the stomach, as well as to decompress, empty and remove gastric acid.

The calibration tube 1 has a terminating element 16 at the second tube end 14. The terminating element 16 is blunt so that no injuries can occur when the second tube end 14 is inserted into the human body. The hollow body 2 has at least one channel 6. The channel 6 extends in the longitudinal direction of the hollow body 2.

The gastric calibration tube 1 has at least one outlet opening 22 in the area of the second tube end 14. In the present case, several outlet openings 22 are provided in the hollow body 2. Furthermore, the terminating element 16 can also have at least one outlet opening 18, which opens into the channel 6.

Medical aids such as syringes, cannulas or devices that introduce or discharge air or liquids into or from the channel 6 of the calibration tube 1 can be connected to the channel 6 at the first tube end 7. A negative pressure can be generated in the channel 6 of the calibration tube 1 for discharge purposes. If a negative pressure is generated in the channel 6, a negative pressure is also present at the outlet openings 22 and 18. In this way, a suction can be performed using the first channel 6.

In FIG. 1a, a connecting device 64 is provided, which is connected to the first tube end, the calibration tube 1 being inserted into the connecting device 64. The connecting device 64 can be used to ensure simple and secure coupling of different medical devices with a wide variety of connection geometries.

At the end of the connecting device facing away from the calibration tube 1, the connecting device for coupling different medical aids has at least one second tapered indentation and at least one tapered recess annularly surrounding the second indentation, so that the different medical aids can be inserted either into the second tapered indentation or into the tapered recess for coupling.

However, the calibration tube according to the invention can also be connected to a medical aid without or with a different connecting device.

FIG. 1a also shows a locking slider 80. This is shown in more detail in FIG. 1b. The same has a passage opening 82, which has a first region 84 through which the calibration tube 1 can be guided without being clamped. The passage opening 82 has a second tapered region 87. When the calibration tube 1 is pressed in the direction of the second tapered region, the calibration tube 1 is compressed and the channel 6 of the calibration tube is closed.

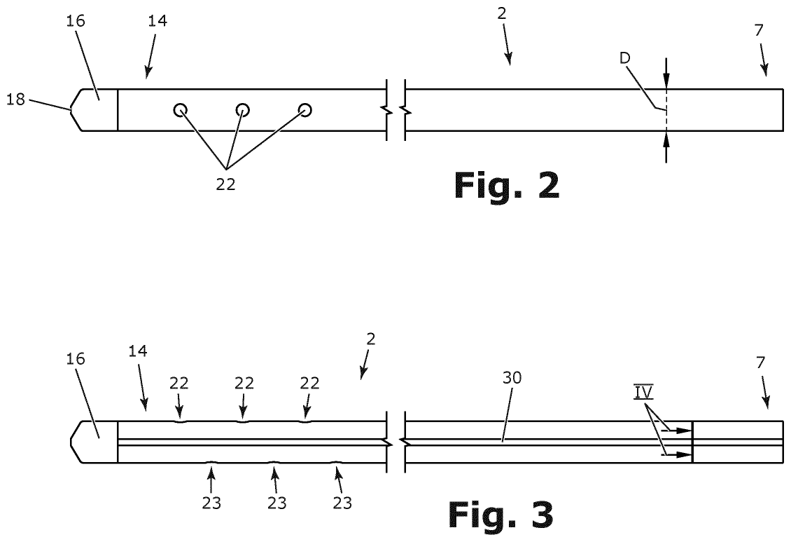

FIG. 2 shows the calibration tube in more detail, the first tube end 7 and the second tube end 14 being shown in more detail for clarification and the middle portion of the hollow body 2 is left out. Furthermore, the connecting device 64 and the locking slider 80 are not shown in FIG. 2.

FIG. 2 shows the outlet openings 22 at the second tube end 14. The terminating element 16 can also be seen. The terminating element 16 also has an outlet opening 18. As shown, the outer diameter of the terminating element 16 can have the same width as the diameter D of the elongate hollow body 2. Both the terminating element 16 and the elongate flexible hollow body 2 can be made of silicone, preferably silicone rubber. The material of the elongate flexible hollow body 2 preferably has a Shore hardness A of between 60 and 100. The Shore hardness A of the terminating element 16 is preferably lower than the Shore hardness A of the flexible hollow body. The terminating element 16 is therefore preferably softer than the hollow body 2

FIG. 3 shows the gastric calibration tube 1 of FIG. 2, but in a different side view. In this side view, it can be seen that at least one color strip 30 can be arranged along the elongate flexible hollow body 2 in the longitudinal direction of the elongate flexible hollow body. Furthermore, it can be seen that first outlet openings 22 can be arranged on a first side of the hollow body 2 in the region of the second tube end 14 and, furthermore, second outlet openings 23 can be arranged on a second side of the hollow body 2 opposite the first side. The first and second outlet openings 22, 23 are preferably arranged offset to each other.

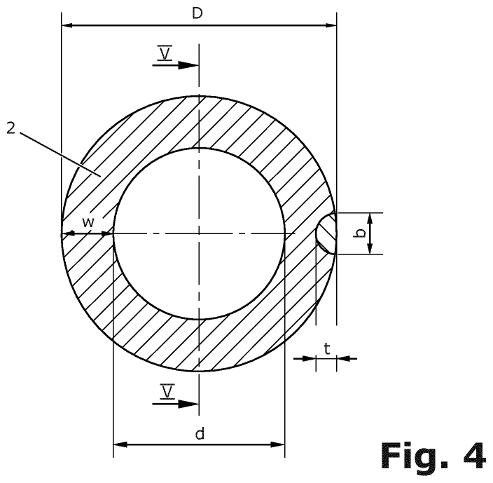

FIG. 4 shows a cross-section through the elongate flexible hollow body 2 of FIG. 3. FIG. 4 shows the outer diameter D of the elongate flexible hollow body and the inner diameter d. The wall thickness w is also shown.

According to the present invention, the ratio between the wall thickness w of the elongate flexible hollow body 2 in relation to the outer diameter D of the elongate flexible hollow body is between 0.1 and 0.25, in particular between 0.16 and 0.20.

FIG. 4 further shows that a color strip 30 can be provided. This is an optional feature. As illustrated, the color strip can be recessed in the elongate flexible hollow body 2 so that the surface of the color strip is flush with the outer surface of the elongate flexible hollow body 2. The color strip has a width b and a thickness t. The thickness t of the color strip is preferably less than the wall thickness w of the hollow body 2, so that the wall of the hollow body 2 is not pierced or broken through.

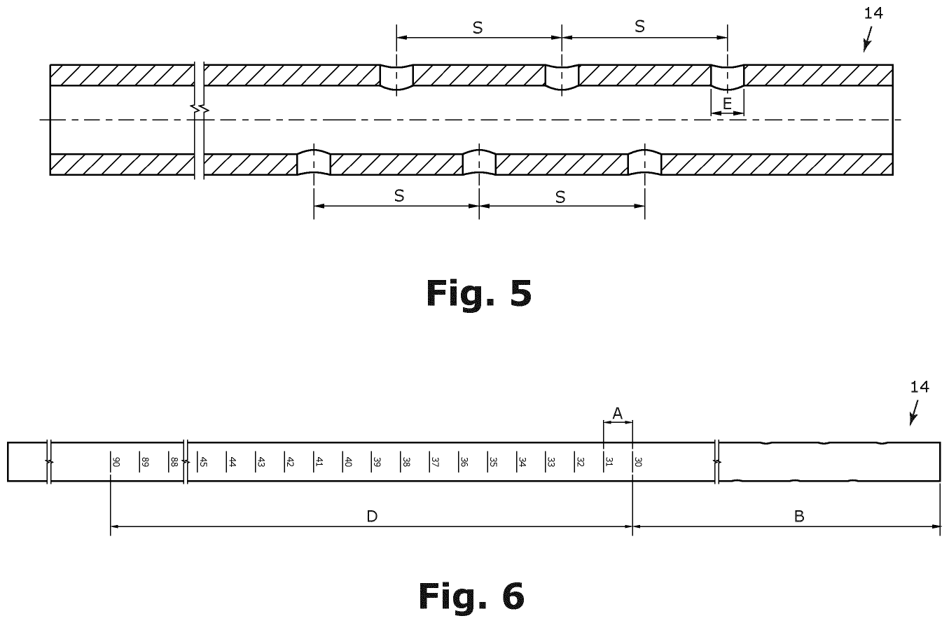

FIG. 5 shows a longitudinal section through the hollow body 2. There it can be seen once again that the first outlet openings 22 and the second outlet openings 23 are arranged offset to each other. It can also be seen that the outlet openings 22 and 23 have the diameter E. The diameter E is preferably between 2 and 6 mm. The distance S between the outlet openings 22 and 23 is preferably between 10 and 30 mm.

The terminating element 16 is formed from a material 20 which preferably comprises barium sulphate and is thus recognizable by X-ray methods.

1-16. (canceled)

17. Gastric calibration tubes comprising
an elongate flexible hollow body which has an outer wall and a first and a second tube end,
wherein the elongate flexible hollow body has at least one opening in the region of the second tube end,
wherein
the ratio between the wall thickness of the elongate flexible hollow body in relation to the outer diameter of the elongate flexible hollow body is between 0.1 and 0.25.

18. The gastric calibration tubes according to claim 17, wherein the elongate flexible hollow body consists of a material which comprises silicone, preferably silicone rubber, the silicone rubber preferably having a Shore hardness A of between 60 and 100.

19. The gastric calibration tubes according to claim 17, wherein the elongate flexible hollow body can have at least such a resistance to kinking that with a tube section of maximum 20 cm in length, the two tube section ends can be brought together and the tube section does not kink.

20. The gastric calibration tubes according to claim 19, wherein the elongate flexible hollow body can have at least such a resistance to kinking that with a tube section of maximum 15 cm in length, the two tube section ends can be brought together and the tube section does not kink.

21. The gastric calibration tubes according to claim 17, wherein the ratio between the wall thickness of the elongate flexible hollow body in relation to the outer diameter of the elongate flexible hollow body is between 0.16 and 0.20.

22. The gastric calibration tubes according to claim 17, wherein outlet openings are provided in the region of the second tube end.

23. The gastric calibration tubes according to claim 17, wherein a terminating element is provided at the second tube end, which terminates the hollow body.

24. The gastric calibration tubes according to claim 23, wherein the end element is blunt.

25. The gastric calibration tubes according to claim 23, wherein the terminating element comprises at least one outlet opening.

26. The gastric calibration tubes according to claim 23, wherein the terminating element has a material comprising radiopaque material, preferably barium sulphate.

27. The gastric calibration tubes according to claim 23, wherein the material from which the terminating element is made is softer than the material from which the elongate flexible hollow body is made.

28. The gastric calibration tubes according to claim 17, wherein the material of which the terminating element is made comprises silicone, preferably silicone rubber.

29. The gastric calibration tubes according to claim 17, wherein at least one color strip is arranged along the elongate flexible hollow body in the longitudinal direction of the elongate flexible hollow body.

30. The gastric calibration tubes according to claim 17, wherein the color strip is recessed in the elongate flexible hollow body so that the surface of the color strip is flush with the outer surface of the elongate flexible hollow body.

31. The gastric calibration tubes according to claim 17, wherein the elongate flexible hollow body has first outlet openings on a first side of the hollow body in the region of the second tube end and second outlet openings on a second side of the hollow body opposite the first side.

32. The gastric calibration tubes according to claim 31, wherein the first and second outlet openings are arranged offset to one another.
